(19) United States
(12) Patent Application Publication
(10) Pub. No.: US 2012/0064155 A1
(43) Pub. Date: Mar. 15, 2012

(54) ORAL PHARMACEUTICAL COMPOSITION FOR USE IN RESPIRATORY DISEASES

(75) Inventors: Raúl García Salgado López, Colonia Anahuac (MX); Juan Aurelio Senosiain Aguilar, Colonia Anahuac (MX)

(73) Assignee: LABORATORIOS SENOSIAIN S.A. DE C.V., Distrito Federal (MX)

(21) Appl. No.: 13/202,743

(22) PCT Filed: Oct. 1, 2009
(86) PCT No.: PCT/IB2009/007023

§ 371 Date: Sep. 23, 2011

(30) Foreign Application Priority Data
Feb. 20, 2009 (MX) MX/A/2009/001925

(51) Int. Cl. A61K 9/52; A61P 27/02; A61P 37/08; A61P 11/02; A61K 31/4545; A61K 31/137

(52) U.S. Cl. 424/457; 514/290; 514/653

(57) ABSTRACT

A modified-release oral pharmaceutical composition in capsules with microspheres contains loratadine, phenylephrine and pharmaceutically acceptable excipients. The composition has immediate bioavailability, with plasmatic concentration values within the therapeutic window with a uniform, continuous release. A method for the production of the composition and a method for treatment as a nasal decongestant and an antihistamine are included.

### FIELD OF THE INVENTION

The subject invention refers to an oral pharmaceutical composition of modified release capsules with coated nuclei that contain an antihistamine drug, a decongestant, and pharmaceutically acceptable vehicles or excipients; as well as to the process of manufacturing said pharmaceutical composition and its use for the treatment as antihistamine, nasal decongestant.

### BACKGROUNDS OF THE INVENTION

In the treatment of congestive respiratory ailments, it is desired that the pharmaceutical compositions act in a prompt and sustained manner. In such way that there are currently existing pharmaceutical compositions with monodrugs and combinations thereof of immediate and modified release that attend said ailments. This invention provides a formulation containing an antihistamine, as loratadine, and a decongestant, phenylephrine, reaching a maximum plasma concentration in less time, as compared to similar formulations, and obtaining a longer sustained therapeutic effect.

Loratadine is a long-acting antihistamine, selective antagonist of peripheral receptors H₁; after its oral administration it is completely absorbed, its half life of plasma elimination is of 9 hours. Its antihistamine effect starts within 30 minutes approximately, continuing during a time between 24 and 28 hours, it binds to plasma proteins in a high percentage (97 to 99%). It mainly metabolizes at the liver and excreted by urine and feces.

Loratadine is an antihistamine prescribed for the treatment and relief of the symptoms of allergic rhinitis (sneezing, watery eyes and Rinorea), allergic dermatitis and conjunctivitis. Loratadine has higher physical stability in lightly acid solutions, in a pH rate among 5.0 and 6.9. Loratadine may be administered, taking into account the recommendation from the doctor, from 2.5 mg up to 20 mg per day.

Phenylephrine is an agonist medication of the a adrenergic receptors, it does not achieve the release of endogenous norepinephrine as the pseudoephedrine, it is useful mainly as nasal decongestant. It is absorbed at the gastrointestinal tract, as effect of the first step, it is metabolized by the monoamine oxidase, both at the intestine and at the liver, also increases the blood pressure, causing a bradycardia effect. It is bound to proteins in 95%, it has an hepatic metabolism and a half life between 2 and 3.5 hours. Given the half life, it is recommended to administer the product to from 4 to 6 times a day, it may be administered, taking into account the recommendation from the doctor, from 10 mg up to 180 mg per day.

Phenylephrine has higher physical stability in a solution at lightly basic pH in a rate between 7.0 and 9.0, has a water solubility of 100 mg/ml, same that, when combining it with a binding agglutinant agent from the type of hydroxypropylmethylcellulose (HPMC) and hydroxymethylcellulose (HMC), propylene-glycol (PEG), polyvinipylirridone or any other similar, a plastic solution is formed, that difficults its accession to the inert nuclei, also referred to in this document as microspheres.

The formulation of the present invention, additionally to loratadine and phenylephrine, may also include another substance with therapeutic activity, same may be an AINE as ibuprofen, acetaminophen, nimesulide, among others.

In the state of the art, there is the patent document WO/2006/135254, that refers to an oral solid composition of immediate release that may be offered in capsules, tablets or syrup. The disclosed composition contains loratadine 2.5 mg and phenylephrine between 8 and 10 mg per unit dose. Said composition is administered, at least, four times per day and is indicated for the treatment of the mucous membrane of the upper respiratory tract congestion.

This invention, that preferably includes loratadine 5 mg and phenylephrine 20 mg per unit dose, is offered in capsules containing coated nuclei; said composition is of immediate and modified release, which allows to obtain the decongestant and antihistamine therapeutic activity in an immediate and sustained manner, thus lowering the intakes to two times per day.

The patent document WO/2007/098128 refers to an aqueous oral composition and/or oral composition in aqueous suspension and its process of manufacturing. This composition preferably contains phenylephrine with a second active, among which, loratadine may be selected. This composition stabilizes at an acid pH between 2 and 5.4.

Unlike this formulation, the present invention particularly refers to a pharmaceutical oral composition in capsules with coated nuclei that contain loratadine and phenylephrine, wherein the formulation being characterized because phenylephrine has a modified release profile; the stability of the system of the formulation is obtained by a design of compartments that separate the release phases of the formulation; besides the fact that during the formulation process, it is in a pH rate between 5 and 7.

The patent document WO/2007/066178 is related to a composition of softgel capsules and it manufacturing process. Said composition contains phenylephrine and, additionally, it may contain a second active (among which there may be an antihistamine as loratadine). The softgel capsule composition comprises an hydrophobe internal nuclei and at least an hygroscopic polyol and, at least, a polyol with low hygroscopicity. It is worth noting that said composition is of immediate release.

Unlike this formulation, the present invention refers to an oral pharmaceutical composition in hardgel capsules with coated nuclei that contain loratadine and phenylephrine. This design provides the feature of being an immediate and modified release formulation.

On the other side, for the treatment of the nasal congestive ailments, there are several pharmaceutical compositions, from which, the most commonly used comprise an antihistamine and a decongestant. Such is the case of the composition of loratadine 5 mg and phenylephrine 30 mg offered as tablets, that is currently commercialized. Said composition is administered at least twice a day.

Unlike this formulation, this invention contains only 20 mg of phenylephrine, that represents two thirds of the contents in the existent commercial formulation of loratadine and phenylephrine, without compromising the antihistamine therapeutic effect and nasal decongestant. This was proved by a bioavailability test, which demonstrated that the formulation of this invention reaches an anticipated plasma concentration, and, at the same time, constant during a time period that allows it to be a modified release formulation. Additionally, when offering a formulation with a lower amount of phenylephrine, it may cause fewer side effects.

### SUBSTANTIATION OF THE INVENTION

In the treatment of respiratory congestive ailments, it is desired that the pharmaceutical compositions act in an immediate and sustained manner with an administration system lowered as much as possible in order to avoid the generation of the treatment detachment.

In this invention, at least two actives are included, a fast response antihistamine (loratadine) and an immediate and long-acting effect decongestant (phenylephrine). It is worth to note that it is possible to add another substance with therapeutic activity, that may be an AINE as ibuprofen, acetaminophen, nimesulide, among others.

The integration of the active ingredients is possible due the design of the formulation that consists of inert nuclei coated by successive coats of excipients, vehicles and active ingredients, which allows for the active ingredients to avoid direct contact among them and also to be released in a modified or immediate manner.

With the design of the formulation of inert nuclei coated by three coats of excipients and active ingredients is achieved by giving a better uniformity and contents release, provides a prompt bioavailability of the active ingredients and gastro-protect, which allows a modified and sustained release.

If considered appropriate, a third active ingredient may be included, whether in the first film or coat and will be of modified release (as the active ingredient with which it is combined), or well, it may be added in the exterior film and coat of immediate release. The order of addition will depend on the features wished to be obtained for releasing the third active ingredient.

In this formulation, a system of excipients is used as binder hydroxypopylmethylcelullose (HPMC), Eudraflex-2 plasticizer and the coating polymer Eudragit S-100, same that create an environment enabled for the coexistence of the active ingredients and that one of them may be released in a modified manner.

On the other hand, by means of a comparative bioavailability test of this invention in capsules with microspheres with a similar formulation with antihistamine and nasal decongestant in tablets, this invention showed having reached a maximum plasma concentration (Cmax) one hour after its administration, which is the half of the time that the compared formulation in tablet uses.

Additionally, the formulation of this invention maintains the plasma concentration during the designed period of modified release. The release of the active ingredient is continuous and constant, in benefit of the patient.

### BRIEF DESCRIPTION OF THE DRAWING

FIG. 1 depicts the results of a bioavailability test, where in the temporal course of the phenylephrine concentration after administration of the pharmaceutical compositions. In the “X” axis time is represented in hours, and in the “Y” axis, the plasma concentration is represented.

### DESCRIPTION OF THE INVENTION

The challenge that the development of this invention faced is obtaining a stable oral pharmaceutical composition, safe and with therapeutic effectiveness that includes at least loratadine and phenylephrine and/or its pharmaceutically acceptable salts.

For generating capsules with coated nuclei, it is necessary to have excipients that serve as vehicle of the active ingredients for being sprayed over the inert nuclei, as well as to confer gastro-protection for its modified release.

In the process of manufacturing the formulation of the pharmaceutical composition of loratadine with phenylephrine, technical problems were detected that precluded the integration of the active ingredients to the inert nuclei. Having in mind the state of the art, the use of a binding agglutinate agent (as HPMC) was foreseen, for adhering the phenylephrine to the inert nuclei. Nevertheless, there were different problems found, one of the most important is the difficulty that arises for its adhesion.

When combining the phenylephrine in solution with a binding agglutinant agent, it forms a plastic solution, same that is impossible to spray over the inert nuclei, as an agglomeration of the product is formed, preventing the adhesion over the microsphere.

Due to technical problems, several binding agglutinate polymers were tested, such as hydroxy-propyl-methyl-cellulose HPMC, hydroxy-methyl-cellulose HMC, poly-viny-pylirridone, poly-ethylen-glycol, among others. These agents were used in the indicated and recommended amounts for the addition of active ingredients, sprinkling it over inert nuclei without obtaining good results, as when it was used in the suggested amounts, the sprinkling solution becomes plastic and is not adhered to the inert nuclei. On the other hand, if the concentration of the binding agglutinating agent is lowered, the obtained solution does not have the degree of adherence so as to fix to the inert nuclei.

From this problem, it was deemed necessary to develop a system that worked as: vehicle for carrying active ingredients and excipients to the inert nuclei; as well as creating a micro-environment that confers stability to the pharmaceutical composition.

For the physicochemical interactions of phenylephrine, it was necessary the design of a system that allows its solubility and adherence to the inert nuclei. This technical problem was solved surprisingly after several assays, with the use of a higher amount of the binding agglutinant agent and when combining it with an agent that modifies the plasticity of the composition (as the Eudtraflex-2), to abate the impossibility of adhesion to the inert nuclei.

The formulation of this invention, additionally is gastroresistant and of modified release, allowing it to reach the release site in the gut. The principal excipient used for this purpose is the coating agent Eudragit S-100.

The formulation of this invention comprises an extended-release capsule containing from 2.5 mg up to 20.0 mg of loratadine and from 10.0 mg up to 180 mg of phenylephrine, or its pharmaceutically acceptable salts, characterized because it comprises microsperes with the following configuration:

a) an inert nuclei coated with a first film or coat made by the 30-70% of the phenylephrine dose and at least an adhesive polymer;

b) a second film or coat made by at least a retardant polymer; and

c) a third film or coat made by 30-70% of the phenylefrine dose, loratadine and at least one adhesive polymer.

wherein, phenylefrine presents modified release and loratadine presents immediate release.

This formulation surprisingly showed that the composition with the association of loratadine and phenylefrine reaches the maximum plasma concentration in a one hour time, which means the half of the time in which it is reached by an existing commercial formulation and that contains the same active ingredients. This was proved when making a comparative bioavailability test made for this formulation.

### Formulations

Following, the formulation and process of manufacturing is described with the oral formulation, same that contains the active ingredients of loratadine and phenylefrine, and/or its pharmaceutically acceptable salts, additionally vehicles or pharmaceutically acceptable salts. The amounts of weight of the active ingredients, vehicles and/or excipients may be used within the ranks of use cited as follows.

Active Ingredients:
Loratadine, it may be administered from 0.17% up to 7.1% in weight of the composition.
Phenylefrine, may be administered from 0.35% up to 28.5% in weight of the composition.

Excipients and/or Vehicles:
Base of inert nuclei that may be of cellulose or sugars, selected from: cellulose, starch, glucose and dextrose among others. This base will confer support to the active ingredients and to the vehicles or excipients of the microsphere.
Binding or agglutinant agent or adhesive polymer, selected from hydroxy-propyl-methyl-cellulose (HPMC), hydroxy-propyl-cellulose (HPC), poly-vinyl-pylirridone, among others. This polymer confers adherence and adhesion and coherence to the microsphere, besides protection to the active ingredient.
Lubricant, selected from starch, talc, calcium carbonate, calcium phosphate, titanium dioxide, among others. It lowers the adherence of the coating polymer, and it helps in making smooth the surface of the microsphere.
Plasticizer, selected from polyethylenglycol, propylenglycol, polyvynil alcohol, Eudraflex-2 or polysorbates, glycerine, phthalate esters, among others. It grants features of resistance, plasticity and improves the quality of the coating film, specially during drying.
Coating polymer or retardant polymer, selected form polyvynil alcohol, derived from cellulose, derived from metacrylates such as: Eudragit L-100 or Eudragit S-100, among others. This component protects the microspheres from the environment and during its pass through the gastrointestinal tract, which provides stability and allows the modified release to happen.

Within the range of the cited excipients, several equivalent excipients and/or mixtures thereof were tested.

In case of deeming appropriate, a third active ingredient may be included, either on the first film or coat, and will be of modified release (as well as the active ingredient with which it is combined), or well, may be added in the film or exterior coat of immediate release.

The order of addition will depend on the features desired to be obtained for releasing the third active ingredient.

Following, you will find representative examples of oral formulations of this invention.

### EXAMPLE 1

### Oral Formulation

In Table 1, there is expressed the oral general formulation of the combination of loratadine and phenylefrine. In Table 2, there are depicted examples of formulations that comprise loratadine and phenylefrine, wherein the amounts in weight of the active ingredients, vehicles and/or excipients may be used within the mentioned ranks of use, without limiting its use.

TABLE 1
ORAL GENERAL FORMULATION.

| | Rank of use. Percentage/ |
|---|---|
| | per each 100 mg of |
| COMPONENTS | composition |
| Loratadine | 0.1-7.1 |
| Phenylefrine | 0.3-28.5 |
| Inert nuclei | 53.4-62.3 |
| Binding Agglutinant Agent | 8.9-11.4 |
| Plasticizer | 5.3-7.1 |
| Lubricant | 2.3-3.0 |
| Coating polymer | 11.4-12.4 |
| Water | Cbp |
| Hardgel capsule | Yes |
| Total Weight | 100% |

TABLE 2
ORAL FORMULATIONS EXAMPLE.

| | Ranks of use in percentage/per each 100 mg | | |
|---|---|---|---|
| | of composition | | |
| Ingredients | Formulation 1 | Formulation 2 | Formulation 3 |
| Loratadine | 0.1 | 1.8 | 7.1 |
| Phenylefrine | 0.3 | 7.1 | 28.5 |
| Inert nuclei | 62.3 | 59.9 | 53.4 |
| HPMC | 11.4 | 10.3 | 8.9 |
| Eudraflex-2 | 7.1 | 6.0 | 5.3 |
| Talc | 3.0 | 2.6 | 2.3 |
| Eudragit S- | 12.4 | 12.1 | 11.4 |
| 100 | | | |
| Water | Cbp | Cbp | Cbp |
| Hardgel | Yes | Yes | Yes |
| capsule | | | |
| Total | 100% | 100% | 100% |

The process for obtaining the microspheres of the invention, is characterized by the fact that over the inert nuclei it is added by sprinkling:

a) a first film or coat made by the 30-70% of the phenylefrine dose and at least one adhesive polymer;

b) a second film or coat made by at least one retardant polymer; and

c) a third film or coat made by the 30-70% of the phenylefrine dose, loratadine and at least one adhesive polymer.

Described below is a form for preparing the composition. The following example illustrates the object of the invention by means of the oral formulation in capsules with microspheres without same being limited for such reason.

Make sure that the materials and equipment correspond to the manufacturing of the formulation. The process for the preparation of the preferred formulation is the following:

1. The compounds of the formulation are weighed.

2. In a vessel, disperse into water a parte of the binding agglutinant agent (between 40 and 45% of the total) and the active ingredient phenylefrine (between 30 and 70% of the total), stir constantly.

3. Load the fluidized bed equipment with the inert nuclei.

4. Apply the mixture of step 2 over the inert nuclei by means of sprinkling and stirring of the fluidized bed equipment.

5. In a vessel, disperse the coating polymer, stabilize the pH.

6. In another vessel, disperse the plasticizer in water.

7. Place the dispersions of steps 5 and 6 in the same vessel.

8. In another vessel, dissolve the lubricant in water.

9. Add the solution of step 8 to the one of step 7.

10. Sprinkle the solution of step 9 to the inert nuclei of item 4.

11. In another vessel, dissolve in water the remaining binding agglutinant agent (between 55 and 60%).

12. Once the solution of step 11 is homogeneous, separate two moieties of the solution. “Solution A”.

13. Add to the homogeneous solution of step 11 the phenylefrine hydrochloride active ingredient (between around 30 and 70% of the total).

14. In the solution A, separated at step 12, the loratadine is dissolved by slow stirring.

15. Add once the mixture of step 14 is homogeneous with the one of step 13. Stir until homogeneous.

16. Apply the solution of item 15 over the coated nuclei of step 10, by sprinkling.

17. Dry the coated nuclei and maintain in the fluidized bed equipment.

18. Fill up the capsules of hardgel with the coated microspheres.

This oral formulation overwent a comparative bioavailability test among the formulation of this invention (capsules with microspheres containing 5 mg of loratadine and 20 mg of phenylefrine) and a comparative formulation (tablets containing 5 mg of loratadine and 30 mg of phenylefrine).

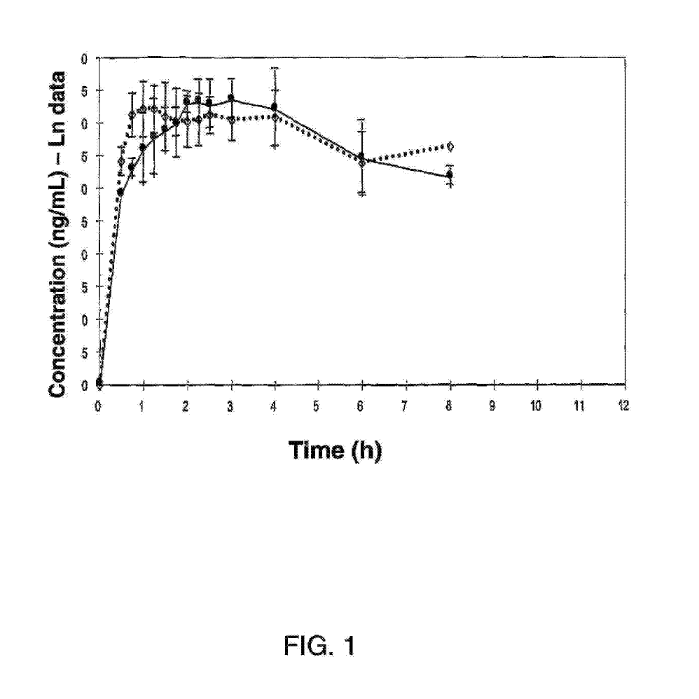

FIG. 1 depicts the results of the bioavailability test, wherein the temporal course of the phenylefrine concentration after the administration of the two pharmaceutical combinations is shown. The test was performed in an aleatory manner, double blind.

Results.

TABLE 3
Results of the comparative
bioavailability test.

| | Phenylefrine descriptive statistics on raw | | |
|---|---|---|---|
| | data (adjusted to TRM*) | | |
| | | | PI |
| | | Present | Formulation/ |
| | | Invention | (comparison |
| | Comparison | formulation | Formulation * |
| Parameter | Formulation | (PI) | 100) |
| ABC | 371.60 ± 148.12 | 344.68 ± 136.00 | 92.76 |
| (ng/ml * hour) | | | |
| Cmax (ng/ml) | 94.18 ± 25.63 | 79.50 ± 17.15 | 84.42 |
| T max (hours) | 2.83 ± 1.40 | 1.56 ± 0.66 | 55.31 |
| T ½ (hours) | 1.99 ± 0.42 | 2.72 ± 0.64 | 136.54 |

*Sampling real time

The present invention contains only 20 mg of phenylefrine, that represents two thirds of the contents of the existing commercial formulation. In the present test, it was shown that the formulation of the present invention reaches an anticipated plasma concentration, it means, one hour before the comparison formulation.

FIG. 1 depicts, in black diamonds, the formulation of the present invention “A” (contains 5 mg of loratadine and 20 mg of phenylefrine), and comparison formulation “B” (contains 5 mg of loratadine and 30 mg of phenylefrine), is depicted in black circles.

Formulation “A” reaches Cmax in 1.56±0.66 hours, while the comparison formulation reaches it in 2.83±1.40 hours.

As it may be seen from FIG. 1, the plasma concentration of the present invention is reached in a lower time than the comparison formulation, with plasma concentration values within the therapeutic window with a uniform and continuous release.

This results are reached with a formulation containing a lower amount of the active ingredient phenylefrine, with respect to the comparison formulation, without affecting the therapeutic effect, substantially enhancing the therapeutic effect, and, furthermore, the presence of side effects may be lowered.

The developed system is non-limitative of including two active ingredients, as it may contain a third narcotic analgesic non-steroidal active ingredient, such as ibuprofen, acetaminophen, nymesulide, among others.

The invention has been sufficiently described so that an average skilled in the art may reproduce and obtain the results that we mentioned in this description. Nevertheless, any person skilled in the art within which this invention falls, may be able to make amendments that are not described in the present application. Nevertheless, if in order to apply these modifications in a determined composition, it is required the matter that is claimed in the following claims, said compositions must be comprised within the scope of the present invention.

1. A long-acting release capsule comprising:
from 2.5 mg up to 20.0 mg of loratadine, or pharmaceutically acceptable salts thereof,
from 10.0 mg up to 180 mg of phenylephrine, or pharmaceutically acceptable salts thereof, and
microspheres with the following configuration:
a) an inert nuclei coated with a first film or coat made by the 30-70% of the phenylephrine dose and at least an adhesive polymer;
b) a second film or coat made by at least a retardant polymer; and
c) a third film or coat made by 30-70% of the phenylephrine dose, loratadine and at least one adhesive polymer,
wherein, phenylephrine presents modified release and loratadine presents immediate release.

2. The long-acting release capsule of claim 1, comprising 5 mg of loratadine, or pharmaceutically acceptable salts thereof, and 20 mg of phenylephrine hydrochloride, or pharmaceutically acceptable salts thereof, per unit dose.

3. The long-acting release capsule of claim 1, wherein the adhesive polymer is hydroxypropylmethylcellulose (HPMC).

4. A method for treating allergic rhinitis, nasal congestion or ocular nasal itching, comprising:
administering the long-acting release capsule of claim 1 to a patient in need thereof.

5. A process for obtaining the microspheres of claim 1, wherein, over the inert nuclei, the following is added by sprinkling:
a) a first film or coat made by the 30-70% of the phenylephrine dose and at least one adhesive polymer;
b) a second film or coat made by at least one retardant polymer; and
c) a third film or coat made by the 30-70% of the phenylephrine dose, loratadine and at least one adhesive polymer.
